(19) United States
(12) Patent Application Publication
(10) Pub. No.: US 2026/0215375 A1
(43) Pub. Date: Jul. 30, 2026

(54) METHOD TO ESTABLISH AN EFFICIENT TISSUE CULTURE SYSTEM FOR RAPID AQUILARIA SINENSIS PROPAGATION

(71) Applicants: Nanjing Forestry University, Jiangsu (CN); EDGENE Biotechnology ( Wuhan ) Co., Ltd., Hubei (CN)

(72) Inventors: Fuyuan ZHU, Jiangsu (CN); Fang DUAN, Hubei (CN); Baoxing HUANG, Jiangsu (CN); Moxian CHEN, Jiangsu (CN); Huijie DENG, Hubei (CN); Chenchen FU, Jiangsu (CN); Tao SONG, Jiangsu (CN); Yixue WU, Jiangsu (CN); Yuzhen ZHAO, Jiangsu (CN)

(73) Assignees: Nanjing Forestry University, Jiangsu (CN); EDGENE Biotechnology ( Wuhan ) Co., Ltd., Hubei (CN)

(21) Appl. No.: 19/035,898

(22) Filed: Jan. 24, 2025

(51) Int. Cl. A01G 2/10; A01C 1/02; A01G 7/04; A01H 4/00

(52) U.S. Cl. CPC A01G 2/10; A01C 1/02; A01G 7/045; A01H 4/005

(57) ABSTRACT

Disclosed is a method to establish an efficient tissue culture system for rapid Aquilaria sinensis propagation. In the present disclosure, tender stem segments of Aquilaria sinensis seedlings or seeds of Aquilaria sinensis harvested in a current year are used as explants. After a plurality of rounds of disinfection, axillary buds are induced to germinate in a sterile environment, and then induced to differentiate into clustered buds, and single buds are then subjected to rooting induction to obtain complete sterile plants. The present disclosure solves the problems of limited ways to acquire materials and slow propagation rate of traditional sowing propagation methods.

### TECHNICAL FIELD

The present disclosure belongs to the technical field of Aquilaria sinensis propagation, and particularly relates to a method to establish an efficient tissue culture system for rapid Aquilaria sinensis propagation.

### BACKGROUND OF RELATED ART

Aquilaria sinensis is an evergreen tree of the genus Aquilaria in the family Thymelaeaceae. Its resinous heartwood is a source of domestic medicinal materials of Aquilaria sinensis, which has extremely high economic value. Its trunk can secrete a substance Aquilaria sinensis to produce traditional Chinese medicine can be produced. Aquilaria sinensis is a traditional precious medicinal material and natural fragrance in China, India, and other Southeast Asian countries, and has functions of downbearing qi, calming the liver, sedating, relieving pain, and the like. In addition, bark fiber of Aquilaria sinensis is well-developed, making it become an excellent raw material for papermaking and artificial cotton, while seeds of Aquilaria sinensis are rich in oil and have significant industrial uses. Aquilaria sinensis is primarily distributed in tropical and subtropical regions across China, including Hainan, Guangdong, Guangxi, Fujian, and Taiwan. However, affected by natural disasters and human destruction for a long period of time, wild resources of Aquilaria sinensis have significantly decreased. In 1987, Aquilaria sinensis was listed as a national rare and endangered plant for second-level protection, and in 1999, it was listed as a national key wild plant for second-level protection. The seeds of Aquilaria sinensis need to be planted immediately after harvesting. Under natural conditions, the seeds of Aquilaria sinensis can only survive for about one month. Fresh seeds are prone to mold when being stored at 4° C., and after one month, vitality of the fresh seeds declines to below 50%. After 55 days, the seeds generally lose their viability, which caused significant inconvenience to related research. Therefore, establishing a rapid propagation system for Aquilaria sinensis is of great importance for the conservation of germplasm resources and the realization of its industrial applications.

In previous studies, mature embryos of Aquilaria sinensis are commonly used as explants to obtain primary sterile seedlings. Alternatively, stem segments with axillary buds can also be used as explants. After disinfection, the axillary buds are induced to germinate in a culture medium to obtain sterile seedlings. Plant tissue culture is a mature technology developed based on the theoretical foundation of plant cell totipotency, and it is not restricted by seasonal time or spatial limitations. The technology can be used for rapid propagation of seedlings, culture of virus-free seedlings, in vitro conservation of germplasm resources, and also lays a solid foundation for biotechnology breeding. Currently, techniques for inducing the formation of Aquilaria sinensis are under continuous improvement, and comparison and screening of different germplasm resources are also underway. Using rapid propagation technology can expand the breeding of Aquilaria sinensis in large quantities in a shorter period of time, and obtain Aquilaria sinensis with stable genetic traits and superior varietal characteristics, which will greatly facilitate the breeding and molecular mechanism research of Aquilaria sinensis.

### SUMMARY OF THE INVENTION

In view of the foregoing problems, the present disclosure provides a method to establish an efficient tissue culture system for rapid Aquilaria sinensis propagation. In the present disclosure, tender stem segments of Aquilaria sinensis seedlings or seeds of Aquilaria sinensis harvested in a current year are used as explants. After a plurality of rounds of disinfection, axillary buds are induced to germinate in a sterile environment, and then induced to differentiate into clustered buds, single buds are then subjected to rooting induction to obtain complete sterile plants, thereby overcoming the problems of limited material acquisition and slow propagation rate of traditional sowing propagation.

Technical solution: In order to solve the above technical problems, the present disclosure provides a method to establish an efficient tissue culture system for rapid Aquilaria sinensis propagation, including the following steps:
(1) culturing explants of Aquilaria sinensis in an axillary bud induction culture medium, and excising axillary buds from stems to obtain sterile buds; and inoculating the sterile buds into a clustered bud induction culture medium for further culture; where the clustered bud induction culture medium is WPM+6-BA 0.1-1 m g/L+NAA 0.1-0.5 m g/L+sucrose 10-30 g/L+Agar 5-10 g/L, pH=5.5-6; and
(2) excising the sterile buds further cultured in the clustered bud induction culture medium in the step (1) from the original buds and transferring them to a rooting and seedling strengthening culture medium for further culture until roots begin to develop from bases of the buds; where the rooting and seedling strengthening culture medium is ½MS+sucrose 20-40 g/L+Agar 5-10 g/L, pH=5.8±1.0.

Specifically, the explants of Aquilaria sinensis in the step (1) includes tender stem segments of Aquilaria sinensis seedlings or seeds of Aquilaria sinensis harvested in a current year.

Specifically, before being cultured, the explants of Aquilaria sinensis are subjected to pre-treatment.

Specifically, steps for the pre-treatment include: cutting each of the explants into small segments having 1-2 axillary buds, rinsing the segments thoroughly with water, conducting a plurality of rounds of disinfection, and air-drying until no obvious water mark on surfaces of the segments after disinfection.

Specifically, steps for the disinfection include: transferring the segments having 1-2 axillary buds into a sterile triangular flask, first soaking the segments in 75% ethanol, then washing twice with sterile water, disinfecting the explants with 0.1% mercuric chloride solution, and washing with sterile water again.

Specifically, the rooting and seedling strengthening culture medium further contains NAA.

Specifically, a culture environment for culturing explants of Aquilaria sinensis in an axillary bud induction culture medium in the step (1) is 26±2° C., with a fluorescent light source, a light intensity of 3000±1000 Lux and a light duration of 16 h per day.

Specifically, the axillary bud induction culture medium in the step (1) is ½MS+6-BA 0.1-0.3 m g/L+NAA 0.1-0.2 m g/L+sucrose 20-40 g/L+Agar 5-10 g/L, pH=5.5-6.

Specifically, a culture environment for culturing the sterile buds in a clustered bud induction culture medium in the step (1) is 26±2° C., with a fluorescent light source, a light intensity of 3000±1000 Lux and a light duration of 16 h per day.

Specifically, a culture environment for further culturing the clustered buds in a rooting and seedling strengthening culture medium in the step (2) is 26±2° C., with a fluorescent light source, a light intensity of 3000±1000 Lux and a light duration of 16 h per day.

The present disclosure has the beneficial effects:

1. In the present disclosure, tender stem segments of Aquilaria sinensis seedlings or seeds of Aquilaria sinensis harvested in a current year are used as explants. After a plurality of rounds of disinfection, axillary buds are induced to germinate in a sterile environment, and then induced to differentiate into clustered buds, single buds are then subjected to rooting induction to obtain complete sterile plants, thereby solving the problems of limited ways to acquire materials and slow propagation rate of traditional sowing propagation methods. Moreover, after multiple experiments, the present disclosure has found that the sterile plants obtained by using the various culture medium formulations and culture conditions of the present disclosure are of higher quality.

2. Traditional sowing propagation can only be performed in June and July each year, while the tissue culture rapid propagation method of the present disclosure allows for indoor production throughout the year.

3. A propagation speed of traditional seed propagation is relatively slow, while the tissue culture rapid propagation method of the present disclosure can yield a large number of offspring in a short period of time, and traits thereof are stable.

### BRIEF DESCRIPTION OF THE DRAWINGS

FIG. 1 is a schematic diagram of sampling of explants of Aquilaria sinensis (left) and top stem segments (right).

FIG. 2 shows germination of an axillary bud in a stem segment of Aquilaria sinensis 30 days after inoculation.

FIG. 3 shows clustered buds of Aquilaria sinensis induced by axillary buds.

FIG. 4 shows rooting and seedling strengthening of sterile seedlings of Aquilaria sinensis.

FIG. 5 shows effect comparison of a clustered bud induction culture medium for sterile seedlings of Aquilaria sinensis.

FIG. 6 shows effect comparison of a rooting and seedling strengthening culture medium for sterile seedlings of Aquilaria sinensis.

### DETAILED DESCRIPTION OF THE EMBODIMENTS

In order to make the objectives, technical solutions and advantages of the present invention clearer, the present invention will be further described in detail below in conjunction with the embodiments. It should be understood that particular embodiments described herein are merely illustrative of the present disclosure, and are not intended to limit the present disclosure.

### Example 1

### 1. Selection of Explants

Green stem segments with a diameter greater than 2 mm at a top of current-year branches of Aquilaria sinensis seedlings were selected as explants (FIG. 1). The explants were induced the germination of axillary buds through an organogenesis pathway to obtain sterile seedlings. Axillary buds at a top of the stem segments had strong growth vitality, were tender with a low degree of lignification, reducing the possibility of contamination with pathogens, making them less likely to be contaminated after inoculation.

### 2. Disinfection and Inoculation of Explants

After the stem segments were cut with scissors, leaves thereon were removed, the stem segments were further cut into small segments of approximately 2 cm in length, and each of the small segments contained 1-2 axillary buds, and removed to soak in water; the explants were rinsed thoroughly with tap water to remove dust and impurities on a surface of the explants, the explants were then collected and put into a container, which was then placed in a laminar flow hood. The explants were transferred into a sterile triangular flask, the explants were first soaked in 75% ethanol for 30 s, washed twice with sterile water, then the explants are disinfected with 0.1% mercuric chloride solution for 8 min and washed them five times with sterile water. The stem segments were dried on filter paper until there was no obvious water mark on surfaces thereof. The dried stem segments were inoculated in an axillary bud induction culture medium with buds facing upwards. The inoculated stem segments together with the axillary bud induction culture medium were then transferred to a culture room for culture in a culture environment of 26±2° C., with a fluorescent light source, a light intensity of 3000 Lux and a light duration of 16 h per day. The observations were continuously performed and records were kept in one month. During the period, contaminated explants were promptly removed, and the culture medium was replaced as needed. The axillary bud induction culture medium (CxI) was: ½ MS+6-BA 0.2 m g/L+NAA 0.1 m g/L+sucrose 30 g/L+Agar 7 g/L, pH=5.8.

### 3. Induction of Clustered Buds

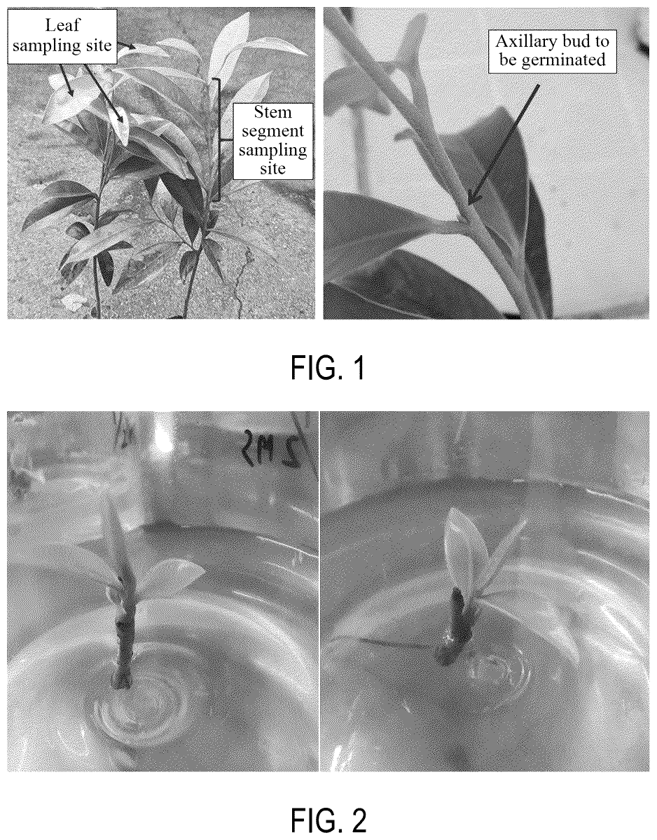

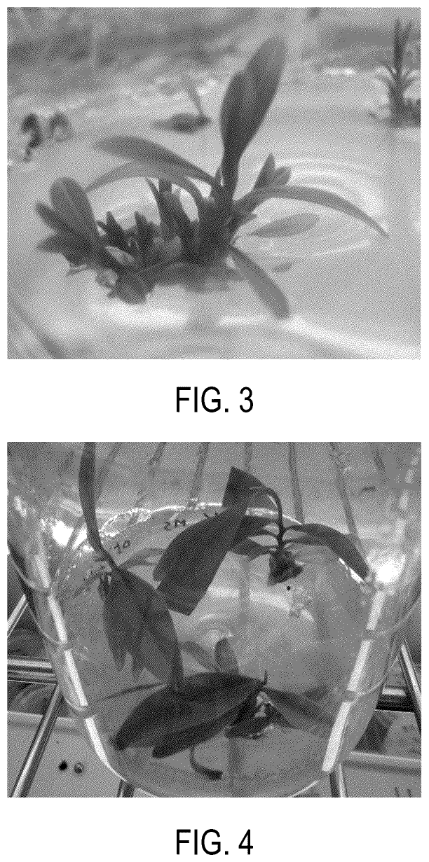

10 days after the stem segments were inoculated, buds between leaf axils began to sprout. After 30 days of culture (FIG. 2), the buds could grow to 1-2 cm, and axillary buds were then excised from the stems to obtain sterile buds, which were then inoculated into a clustered bud induction culture medium for further culture in a culture environment of 26±2° C., with a fluorescent light source, a light intensity of 3000 Lux and a light duration of 16 h per day. After 45 days, bases of the sterile buds began to swell into lumps, and clustered buds emerged from swollen areas, with each lump containing 3-8 buds (FIG. 3). The clustered bud induction culture medium (CxB-1) was: WPM+6-BA 0.5 m g/L+NAA 0.2 m g/L+sucrose 20 g/L+Agar 7 g/L, pH=5.8.

### 4. Rooting and Seedling Strengthening of Sterile Buds

The induced clustered buds continued to be cultured under light. After the clustered buds grew to over 2 cm, single buds were excised from the clustered buds and transferred to a rooting and seedling strengthening culture medium for further culture in a culture environment of 26±2° C., with a fluorescent light source, a light intensity of 3000 Lux and a light duration of 16 h per day. After about 45 days, roots begin to develop from bases of the buds (FIG. 4). The rooting and seedling strengthening culture medium (CxR-1) was: ½ MS+sucrose 30 g/L+Agar 7 g/L, pH=5.8.

### Example 2

### 1. Selection of Explants

Seeds of Aquilaria sinensis harvested in a current year were selected as explants, and germinated in a culture medium after direct disinfection to obtain sterile seedlings. The sterile seedlings obtained from seed germination could directly develop root systems, which grew rapidly, with thick and dark green leaves and robust stems.

### 2. Disinfection and Inoculation of Explants

Seeds harvested in the current year were selected to removing empty seeds, shrunken seeds and seeds with surface mold, seed coats were carefully peeled off, embryos inside are preserved and soaked in water. After preparation, embryo surfaces were rinsed thoroughly with tap water to remove dust and impurities, the embryos were collected in a container, which was then placed in a laminar flow hood. The explants were transferred into a sterile triangular flask, the explants were first soaked in 75% ethanol for 30 s, washed twice with sterile water, then the embryos are disinfected with 0.1% mercuric chloride solution for 8 min, washed them five times with sterile water, and dried on filter paper until there was no obvious water mark on surfaces thereof. The dried embryos were directly inoculated onto a germination culture medium. In order to minimize cross-contamination, the germination culture medium used for inoculation was dispensed into small dishes with a diameter of 60 mm, and 9-12 embryos were inoculated in each dish; embryo tips were inserted downward into a culture medium for dark culture with a culture environment at 26±2° C. The observations were continuously performed and records were kept in 14 days, contaminated embryos were removed promptly, and a new culture medium was replaced as needed. 14 days after inoculation, embryos with normal germination were transferred to a germination culture medium in a tissue culture bottle for further culture until they became seedlings. The germination culture medium C×S was: MS+sucrose 30 g/L+Agar 7 g/L pH=5.8.

### 3. Induction of Clustered Buds

90 days after embryo inoculation, the sterile seedlings were basically well developed, with a height of 6-8 cm, in which case, the sterile seedlings were pulled out from the tissue culture bottle, root systems of the seedlings were removed, and all leaves were trimmed off to keep stem segments only. The stem segments were cut into small sections of about 2 cm in length and inserted into a clustered bud induction culture medium for continuous culture in a culture environment of 26±2° C., with a fluorescent light source, a light intensity of 3000 Lux and a light duration of 16 h per day. After 45 days, bases of sterile buds at the stem segments began to swell into lumps, and clustered buds emerged from swollen areas, with each lump containing 3-8 buds. The clustered bud induction culture medium (CxB-1) was: WPM+6-BA 0.5 m g/L+NAA 0.2 m g/L+sucrose 20 g/L+Agar 7 g/L, pH=5.8.

According to statistics, an induction rate of clustered buds from the stem segments using the method of this example was 100%, with a propagation coefficient of 3.14. The induced clustered buds grew fast, and had dark green leaves, without any malformation.

### 4. Rooting and Seedling Strengthening of Sterile Buds

The induced clustered buds continued to be cultured under light. After the clustered buds grew to over 2 cm, single buds were excised from the clustered buds and transferred to a rooting and seedling strengthening culture medium for further culture in a culture environment of 26±2° C., with a fluorescent light source, a light intensity of 3000 Lux and a light duration of 16 h per day. After about 45 days, roots begin to develop from bases of the buds. The rooting and seedling strengthening culture medium (CxR-2) was ½ MS+sucrose 30 g/L+NAA 1.0 m g/L+Agar 7 g/L, pH=5.8.

According to statistics, a rooting rate of sterile buds using the method of this example was 60%. The root system was numerous and strong, grew radially, with an average number of 13.6 roots per single bud.

### Comparative Example 1

Differences of Comparative Example 1 from Example 1 was that the clustered bud induction culture medium (CxB-2) used by Comparative Example 1 was: WPM+6-BA 0.5 m g/L+sucrose 20 g/L+Agar 7 g/L, pH=5.8

TABLE 1
Comparison of clustered bud induction culture medium
for sterile seedlings of Aquilaria sinensis

| | | | | Propagation |
|---|---|---|---|---|
| | | | | coefficient |
| | | | | (Number of |
| | | Number of | Number of | clustered buds/ |
| | Number of | buds | clustered | Number of |
| Name | inoculations | germinated | buds | buds germinated) |
| CxB-1 | 22 | 22 | 69 | 3.14 |
| CxB-2 | 17 | 16 | 42 | 2.63 |

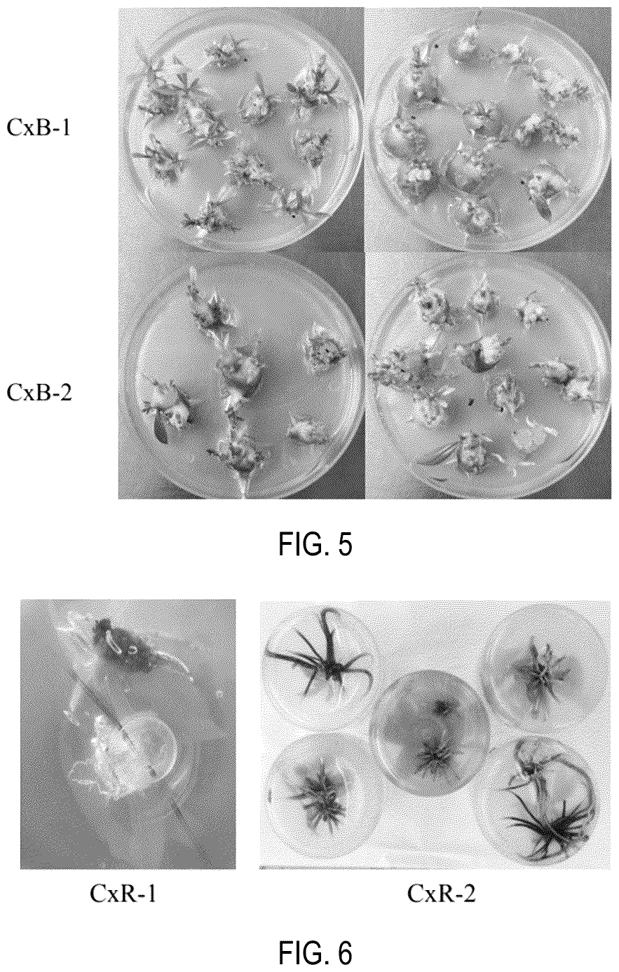

According to statistics, an induction rate of clustered buds using the method of Comparative Embodiment 1 was 94.1% (16/17), with a propagation coefficient of 2.63. The induced clustered buds grew fast, and had dark green leaves, and few buds had malformation. Compared with the C×B-1 culture medium used in Example 1, an induction rate of clustered buds of C×B-1 culture medium was 100%, a number of clustered buds was 1.64 times that of C×B-2 culture medium, and the propagation coefficient was 3.14 (FIG. 5, Table 1).

### Comparative Example 2

Differences of Comparative Example 2 from Example 2 was that: the rooting and seedling strengthening culture medium (CxR-1) used by Comparative Example 2 was: ½ MS+sucrose 30 g/L+Agar 7 g/L, pH=5.8.

TABLE 2
Comparison of rooting and seedling strengthening culture
medium for sterile seedlings of Aquilaria sinensis

| | | | | Average |
|---|---|---|---|---|
| | | Number | | number of |
| | Number of | of roots | Rooting | roots |
| | inoculations | germinated | rate | germinated |
| CxR-1 | 30 | 4 | 13.33% | 1.8 |
| CxR-2 | 30 | 18 | 60.00% | 13.6 |

According to statistics, a rooting rate of sterile buds using the method of this comparative example was 13.3%. The root system was small, short and thin, and an average number of roots germinated per bud was 1.8, which was much lower than the effect of the CxR-2 culture medium used in Example 2 (FIG. 6, Table 2).

1. A method to establish an efficient tissue culture system for rapid Aquilaria sinensis propagation, characterize that, comprising the following steps:
(1) culturing explants of Aquilaria sinensis in an axillary bud induction culture medium, and excising axillary buds from stems to obtain sterile buds; and inoculating the sterile buds into a clustered bud induction culture medium for further culture; wherein the clustered bud induction culture medium is woody plant medium (WPM)+6-benzylamonipurine (6-BA) 0.1-1 mg/L+1-naphthaleneacetic acid (NAA) 0.1-0.5 mg/L+sucrose 10-30 g/L+Agar 5-10 g/L, pH=5.5-6; and
(2) excising the sterile buds further cultured in the clustered bud induction culture medium in the step (1) from original buds, and transferring the sterile buds to a rooting and seedling strengthening culture medium for further culture until roots begin to develop from bases of the buds; the rooting and seedling strengthening culture medium contains ½ Murashige and Skoog (MS) medium+sucrose 20-40 g/L+Agar 5-10 g/L, pH=5.8±1.0.

2. The method to establish an efficient tissue culture system for rapid Aquilaria sinensis propagation according to claim 1, wherein the explants of Aquilaria sinensis in the step (1) comprises tender stem segments of Aquilaria sinensis seedlings or seeds of Aquilaria sinensis harvested within the same year as the culturing.

3. The method to establish an efficient tissue culture system for rapid Aquilaria sinensis propagation according to claim 1, wherein before being cultured, the explants of Aquilaria sinensis are subjected to a pre-treatment.

4. The method to establish an efficient tissue culture system for rapid Aquilaria sinensis propagation according to claim 3, wherein steps for the pre-treatment comprise: cutting each of the explants into small segments having 1-2 axillary buds, rinsing the segments thoroughly with water, conducting a plurality of rounds of disinfection, and air-drying until no obvious water mark on surfaces of the segments after the disinfection.

5. The method to establish an efficient tissue culture system for rapid Aquilaria sinensis propagation according to claim 4, wherein steps for the disinfection comprise: first soaking the segments having 1-2 axillary buds in 75% ethanol, followed by washing with sterile water, disinfecting the explants with a mercuric chloride solution, and followed by washing with sterile water.

6. The method to establish an efficient tissue culture system for rapid Aquilaria sinensis propagation according to claim 1, wherein a culture environment for culturing the explants of Aquilaria sinensis in the axillary bud induction culture medium in the step (1) is 26±2° C., with a fluorescent light source, a light intensity of 3000±1000 Lux and a light duration of 16 h per day.

7. The method to establish an efficient tissue culture system for rapid Aquilaria sinensis propagation according to claim 1, wherein the axillary bud induction culture medium in the step (1) is ½ Murashige and Skoog (MS) medium+6-benzylamonipurine (6-BA) 0.1-0.3 mg/L+1-naphthaleneacetic acid (NAA) 0.1-0.2 mg/L+sucrose 20-40 g/L+Agar 5-10 g/L, pH=5.5-6.

8. The method to establish an efficient tissue culture system for rapid Aquilaria sinensis propagation according to claim 1, wherein the rooting and seedling strengthening culture medium further contains 1-naphthaleneacetic acid (NAA).

9. The method to establish an efficient tissue culture system for rapid Aquilaria sinensis propagation according to claim 1, wherein a culture environment for culturing the sterile buds in a clustered bud induction culture medium in the step (1) is 26±2° C., with a fluorescent light source, a light intensity of 3000±1000 Lux and a light duration of 16 h per day.

10. The method to establish an efficient tissue culture system for rapid Aquilaria sinensis propagation according to claim 1, wherein a culture environment for further culturing the clustered buds in a rooting and seedling strengthening culture medium in the step (2) is 26±2° C., with a fluorescent light source, a light intensity of 3000±1000 Lux and a light duration of 16 h per day.
